(19) United States
(12) Patent Application Publication
(10) Pub. No.: US 2016/0160264 A1
(43) Pub. Date: Jun. 9, 2016

(54) DIAGNOSTIC IN VITRO METHOD

(71) Applicants: UNIVERSITÄT ZU KÖLN, Köln (DE); ROBERT BOSCH GESELLSCHAFT FÜR MEDIZINISCHE FORSCHUNG MBH, Stuttgart (DE)

(72) Inventors: Gunter SCHWARZ, Niederkassel (DE); Julika CSASZAR, Koln (DE); Elke SCHAEFFELER, Stuttgart (DE); Matthias SCHWAB, Stuttgart (DE)

(21) Appl. No.: 14/905,661

(22) PCT Filed: Jul. 14, 2014
(86) PCT No.: PCT/EP2014/064999

§ 371 Date: Jan. 15, 2016

(30) Foreign Application Priority Data
Jul. 18, 2013 (DE) 10 2013 011 995.2

(51) Int. Cl. C12Q 1/48; C12Q 1/26

(52) U.S. Cl. CPC C12Q 1/48; C12Q 1/26; G01N 2333/91017; G01N 2333/90212; G01N 2800/50; G01N 2800/52; G01N 2800/7028

(57) ABSTRACT

Disclosed is a diagnostic in vitro method for determining the activity of thiopurine S-methyltransferase (TPMT) in individuals to be examined, characterized in that the content of urothione and/or jukathione in body fluids is determined.

Also disclosed is a diagnostic in vitro method for determining the TPMT activity in cellular extracts of test subjects of any origin, characterized in that molybdenum cofactor (Moco) and/or decomposition stages of Moco are used as substrates for the reaction and the formed quantity of jukathione and/or urothione is determined.

Finally, a diagnostic in vitro method is disclosed, wherein urothione and/or jukathione serves as a biomarker for an increased susceptibility with regard to one or more particular diseases, which is determined in body fluids and/or cellular extracts.

The invention relates to an in vitro method for determining the enzyme activity of thiopurine S-methyltransferase in body fluids. In particular, the invention relates to such a method in which the urothione content and/or the jukathione content is determined in vitro in plasma, serum and/or urine in order to determine the enzyme activity of thiopurine S-methyltransferase. Further, the invention relates to a diagnostic in vitro method using urothione and/or jukathione as a biomarker.

### BACKGROUND

Thiopurine S-methyltransferase (hereinafter mostly referred to as TPMT) is a key enzyme in the drug metabolism in the treatment of various forms of leukemia and chronic inflammatory diseases. TPMT transforms cytostatic drugs, such as 6-mercaptopurine, into the clinically inactive methylated forms (e.g. 6-methylmercaptopurine), which are then excreted renally. In spite of the pharmacological significance of TPMT, which is presented below, its physiological substrate and the function of TPMT in the human metabolism is unknown.

Thiopurines, such as 6-thioguanine (6-TG), 6-mercaptopurine (6-MP) or azathioprine (AZA), are purine antimetabolites that are widely used in the treatment of leukemia, such as acute lymphoblastic leukemia, autoimmune diseases (e.g. Crohn's disease, rheumatoid arthritis), and in recipients of organ transplants. They are analogs of the naturally occurring nucleic acid base guanine. Other indications for thiopurines are, for instance, non-Hodgkin lymphoma, ulcerative colitis, polycythemia vera, psoriatic arthritis, various autoimmune diseases, including systemic lupus erythematosus, Behçet's disease, other forms of vasculitis, autoimmune hepatitis, atopic dermatitis, myasthenia gravis, neuromyelitis optica (Devic's disease), restrictive lung disease.

TPMT is an enzyme which methylates thiopurine compounds. More specifically, TPMT catalyzes the S-methylation of the thiopurine agents. The methyl donor is S-adenosyl-L-methionine, which in turn is transformed into S-adenosyl-L-homocysteine by the reaction. That is, TPMT metabolizes thiopurine agents using S-adenosyl-L-methionine as an S-methyl donor while forming S-adenosyl-L-homocysteine as a byproduct.

Genetic polymorphisms that affect the enzymatic activity of TPMT correlate with the response and the side effects of thiopurine drugs in the treatment of patients.

Thiopurine S-methyltransferase (TPMT; EC 2.1.1.67) is a cytosolic enzyme that catalyzes the S-methylation of thiopurine drugs, such as azathioprine (AZA) and 6-mercaptopurine (6-MP). The enzyme activity shows itself to be extremely variable, with about 1 in 200 Caucasians having a complete deficiency, while 11% exhibit an intermediary and 89% a normal TPMT activity in red blood cells (RBCs) (Schaeffeler et al. 2004). In addition, 1-2% of Caucasians deviate from this trimodal distribution and exhibit a very high level of TPMT activity (Schaeffeler et al. 2004). Since thiopurine-based immunosuppressives afford only a relatively limited therapeutic range, the TPMT polymorphism influences the ratio between the two methylated metabolites 6-methylmercaptopurine (methylated ribonucleotide) and thioguanine nucleotide (so-called 6-TGN) significantly, and thus determines the effectiveness and toxicity of the thiopurine therapy in patients with inflammatory bowel diseases, acute lymphoblastic pediatric leukemia and other diseases (summarized in Teml et al. 2007). For example, individuals with a reduced or lacking TPMT activity haven an increased risk for myelosuppression during therapy with the standard dose of azathiopurine, or 6-MP, because excessively high 6-TGN levels are present in these cases. Therefore, these patients require an individually adapted dosage in their thiopurine treatment (Kaskas et al Gut 2002).

In contrast, individuals with very high levels of TPMT activity are in danger of developing resistances to therapy (due to 6-TGN levels that are too low), because an intensive methylation of thiopurine metabolites occurs in these cases and the active 6-TGN metabolites are formed in smaller amounts for this reason. In addition, liver damage may be caused by high 6-methylmercaptopurine ribonucleotide levels (Dubinsky et al. 2000, Nygaard et al. CPT 2008). Pediatric patients with acute lymphoblastic leukemia and high levels of TPMT activity are subject to an increased risk of relapse under standard treatment conditions (Stanulla et al. 2005).

The molecular fundamentals of TPMT deficiency are well understood. TPMT constitutes one of the few examples in which a pharmacogenetic phenomenon was translated into generally recognized diagnostic routine tests for the adjustment of optimum thiopurine doses. These recommendations were recently updated (Relling et al. 2013), even though the endogenous substrate of TPMT is still not known today.

TPMT activity is generally determined in red blood cells (RBCs). Cut-off values for very low, intermediary and normal activities that are strongly dependent upon the measuring method used were defined (e.g. radiochemical measurement (Weinshilboum et al. 1978) or HPLC-based methods (Kroplin et al. 1998)).

In the 1980s, the genetic foundations of TPMT deficiency were laid (Weinshilboum et al. 1980). Today, 27 single nucleotide polymorphisms (SNP) associated with altered TPMT activities are described (Appell et al. 2013). The co-inventors Dr. Schaeffeler and Dr. Schwab investigated the TPMT genotype-phenotype correlation in a large cohort (1222 persons) of healthy, voluntary test subjects. They were able to confirm the trimodal distribution of the TPMT phenotypes and found a TPMT deficiency in 0.6% (1:180) of the individuals, an intermediary level of TPMT activity in 10.2%, and normal or high levels in 89.2% (Schaeffeler et al Pharmcogenetics 2004).

The concordance between the TPMT genotypes and phenotypes is 98.4% with a sensitivity and specificity for positive and negative predictions of greater than 90% when using the genetic analysis for predicting the correct TPMT phenotype. Nevertheless, there are significant limitations in the activity determination in RBCs and the genetic examination of TPMT in the clinical routine: (1) The determination of the correct TPMT phenotype in patients that received blood transfusions within the last 6-8 weeks due to disease-related anemia is subject to a high risk of misclassification (Cheung et al. 2003; Ford et al. 2004, Schwab et al. 2001). Therefore, further genetic analysis is of the essence in all these cases. (2) In daily routine, the genetic analysis of the TPMT genotype is not carried out by means of sequencing techniques and therefore detects only the frequent mutations that are already known. Therefore, a misclassification due to rarely occurring alleles that are not analyzed may occur.

For the above reasons, the identification of an endogenous substrate that is methylated exclusively by TPMT is of the greatest significance and of great value for improved clinical diagnostics. Such a direct biomarker would remove all of the above-mentioned limitations and would be detectable not only in the blood, but also in urine, which would significantly simplify clinical diagnostics.

Since the TPMT analysis is necessary in the routine treatment of children with acute lymphoblastic leukemia treated with 6-MP (Relling et al. 2013), a simpler urine analysis in this group of patients would be very advantageous indeed. Furthermore, approximately 1-2% of the individuals are characterized by an increased TPMT activity; the underlying genetic cause is still not known today. The determination of the endogenous reaction product of TPMT would also efficiently identify patients with very high levels of TPMT activity (Schaeffeler et al. 2004) and thus reduce or remove the risk of a poorer response of the azathioprine or 6-MP therapy due to a dosage that is too low.

Furthermore, TPMT deficiency is associated with the toxicity of cisplatin, and therefore, a genetic analysis of TPMT is also recommended prior to a cisplatin treatment (Ross et al. 2009). These patients would also benefit from a simple and fast determination of the endogenous TPMT product.

Because of the risk of side effects, pediatric leukemia patients, prior to therapy, are examined with respect to their TPMT activity by means of genetic and enzymatic analysis. These tests involve a lot of effort and, at present, are therefore routinely carried out mostly only in particularly endangered patient groups, e.g. in pediatric patients, whereas other patient groups, such as patients with chronic inflammatory diseases, are most frequently treated with suboptimum doses at first, in order to avoid toxicities. By increasing the dose in, amongst others, patients with a reduced and non-lacking TPMT activity, one gradually approaches empirically the maximum thiopurine dose tolerated by the patient. In the process, there is, on the one hand, always the danger of severe or even lethal side effects due to overdosing, and on the other hand, there is the danger of underdosing, which jeopardizes the success of the treatment.

### NON-PATENT LITERATURE

1. Appell M L, Berg J, Duley J, Evans W E, Kennedy M A, Lennard L, Marinaki T, McLeod H L, Relling M V, Schaeffeler E, Schwab M, Weinshilboum R, Yeoh A E, McDonagh E M, Hebert J M, Klein T E, Coulthard S A. Nomenclature for alleles of the thiopurine methyltransferase gene. Pharmacogenet Genomics. 2013 April; 23(4):242-8. doi: 10.1097/FPC.0b013e32835f1cc0.
2. Stanulla M, Schaeffeler E, Möricke A, Coulthard S A, Cario G, Schrauder A, Kaatsch P, Dördelmann M, Welte K, Zimmermann M, Reiter A, Eichelbaum M, Riehm H, Schrappe M, Schwab M. Thiopurine methyltransferase genetics is not a major risk factor for secondary malignant neoplasms after treatment of childhood acute lymphoblastic leukemia on Berlin-Frankfurt-Münster protocols. Blood. 2009 Aug. 13; 114(7):1314-8. doi: 10.1182/blood-2008-12-193250. Epub 2009 Jun. 17.
3. Teml A, Schaeffeler E, Schwab M. Pretreatment determination of TPMT—state of the art in clinical practice. Eur J Clin Pharmacol. 2009 March; 65(3):219-21. doi: 10.1007/s00228-009-0618-7. Epub 2009 Feb. 7.
4. Schaeffeler E, Zanger U M, Eichelbaum M, Asante-Poku S, Shin J G, Schwab M. Highly multiplexed genotyping of thiopurine s-methyltransferase variants using MALD-TOF mass spectrometry: reliable genotyping in different ethnic groups. Clin Chem. 2008 October; 54(10):1637-47. doi: 10.1373/clinchem.2008.103457. Epub 2008 Aug. 7.
5. Teml A, Schaeffeler E, Herrlinger K R, Klotz U, Schwab M. Thiopurine treatment in inflammatory bowel disease: clinical pharmacology and implication of pharmacogenetically guided dosing. Clin Pharmacokinet. 2007; 46(3):187-208.
6. Dilger K, Schaeffeler E, Lukas M, Strauch U, Herfarth H, Müller R, Schwab M. Monitoring of thiopurine methyltransferase activity in postsurgical patients with Crohn's disease during 1 year of treatment with azathioprine or mesalazine. Ther Drug Monit. 2007 February; 29(1):1-5.
7. Schaeffeler E, Eichelbaum M, Reinisch W, Zanger U M, Schwab M. Three novel thiopurine S-methyltransferase allelic variants (TPMT*20, *21, *22)—association with decreased enzyme function. Hum Mutat. 2006 September; 27(9):976.
8. Stanulla M, Schaeffeler E, Flohr T, Cario G, Schrauder A, Zimmermann M, Welte K, Ludwig W D, Bartram C R, Zanger U M, Eichelbaum M, Schrappe M, Schwab M. Thiopurine methyltransferase (TPMT) genotype and early treatment response to mercaptopurine in childhood acute lymphoblastic leukemia. JAMA. 2005 Mar. 23; 293(12):1485-9.
9. Schaeffeler E, Fischer C, Brockmeier D, Wernet D, Moerike K, Eichelbaum M, Zanger U M, Schwab M. Comprehensive analysis of thiopurine S-methyltransferase phenotype-genotype correlation in a large population of German-Caucasians and identification of novel TPMT variants. Pharmacogenetics. 2004 July; 14(7):407-17.
10. Schaeffeler E, Stanulla M, Greil J, Schrappe M, Eichelbaum M, Zanger U M, Schwab M. A novel TPMT missense mutation associated with TPMT deficiency in a 5-year-old boy with ALL. Leukemia. 2003 July; 17(7):1422-4.
11. Kaskas B A, Louis E, Hindorf U, Schaeffeler E, Deflandre J, Graepler F, Schmiegelow K, Gregor M, Zanger U M, Eichelbaum M, Schwab M. Safe treatment of thiopurine S-methyltransferase deficient Crohn's disease patients with azathioprine. Gut. 2003 January; 52(1):140-2.
12. Schwab M, Schaeffeler E, Marx C, Zanger U, Aulitzky W, Eichelbaum M. Shortcoming in the diagnosis of TPMT deficiency in a patient with Crohn's disease using phenotyping only. Gastroenterology. 2001 August; 121 (2):498-9.
13. Schaeffeler E, Lang T, Zanger U M, Eichelbaum M, Schwab M. High-throughput genotyping of thiopurine S-methyltransferase by denaturing HPLC. Clin Chem. 2001 March; 47(3):548-55.
14. Dubinsky M C, Lamothe S, Yang H Y et al. Pharmacogenomics and metabolite measurement for 6-mercaptopurine therapy in inflammatory bowel disease. Gastroenterology 2000 April; 118 (4): 705-13.
15. Nygaard U, Toft N, Schmiegelow K. Methylated metabolites of 6-mercaptopurine are associated with hepatotoxicity. Clin Pharmacol Ther 2004 April; 75 (4): 274-81.
16. Relling M V, Gardner E E, Sandborn W J, Schmiegelow K, Pui C H, Yee S W, Stein C M, Carrillo M, Evans W E, Hicks J K, Schwab M, Klein T E. Clinical pharmacogenetics implementation consortium guidelines for thiopurine methyltransferase genotype and thiopurine dosing: 2013 update. Clin Pharmacol Ther. 2013 April; 93(4):324-5. doi: 10.1038/clpt.2013.4. Epub 2013 Jan. 17. No abstract available.
17. Weinshilboum R M, Sladek S L. Mercaptopurine pharmacogenetics: monogenic inheritance of erythrocyte thiopurine methyltransferase activity. Am J Hum Genet 1980 September; 32 (5): 651-62.
18. Kroplin T, Weyer N, Gutsche S et al. Thiopurine S-methyltransferase activity in human erythrocytes: a new HPLC method using 6-thioguanine as substrate. Eur J Clin Pharmacol 1998 May; 54 (3): 265-71.
19. Weinshilboum R M, Raymond F A, Pazmino P A. Human erythrocyte thiopurine methyltransferase: radiochemical microassay and biochemical properties. Clin Chim Acta 1978 May; 85 (3): 323-33.
20. Cheung S T, Allan R N. Mistaken identity: misclassification of TPMT phenotype following blood transfusion. Eur J Gastroenterol Hepatol 2003 November; 15 (11): 1245-7.
21. Ford L, Prout C, Gaffney D et al. Whose TPMT activity is it anyway? Ann Clin Biochem 2004 November; 41 (Pt 6): 498-500.
22. Ross C J, Katzov-Eckert H, Dubé M P, Brooks B, Rassekh S R, Barhdadi A, Feroz-Zada Y, Visscher H, Brown A M, Rieder M J, Rogers P C, Phillips M S, Carleton B C, Hayden M R; CPNDS Consortium. Genetic variants in TPMT and COMT are associated with hearing loss in children receiving cisplatin chemotherapy. Nat Genet. 2009 December; 41 (12):1345-9. doi: 10.1038/ng.478. Epub 2009 Nov. 8. Erratum in: Nat Genet. 2013 May; 45(5):578.
23. Vasiliades, J. (1976). Reaction of alkaline sodium picrate with creatinine: I. Kinetics and mechanism of formation of the mono-creatinine picric acid complex. Clin Chem 22, 1664-1671.
24. Goto, M., Sakurai, A., Ohta, K., and Yamakami, H. (1969). Die Struktur des Urothions. J. Biochem. 65:611-620.

### DISCLOSURE OF INVENTION

Thus, there is a demand for a simple, cost-effective and fast method for determining the TPMT activity in the respective patient in order to be able to determine an effective thiopurine dose without the occurrence of side effects during the therapy.

This problem is solved by the method for determining the enzyme activity of TPMT defined in claim 1. Preferred embodiments are presented in the dependent claims. Furthermore, protection is sought for a diagnostic in vitro method using urothione and/or jukathione as a biomarker for an increased susceptibility with regard to one or more particular diseases.

This invention is based upon the insight of the inventors that TPMT catalyzes an essential step in the catabolism of the molybdenum cofactor (Moco).

### BRIEF DESCRIPTION OF THE FIGURES

FIG. 1 is an HPLC chromatogram of the analysis of urothione after the incubation of kidney extract (Kidney) with Moco (MPT), kidney extract (Kidney) with S-adenosylmethionine (SAM), Moco (MPT) with S-adenosylmethionine (SAM), and kidney extract (Kidney) with Moco (MPT) and S-adenosylmethionine (SAM).

FIG. 2 is an HPLC chromatogram of the analysis of urothione after the incubation of recombinant TPMT with different quantities of Moco (MPT) in the absence and presence of alkaline phosphatase (+AP).

FIG. 3 illustrates steady state kinetics with 3 ng/μL purified TPMT and Moco (A) or 6-mercaptopurine (B) as a substrate. (A) Kinetics of the urothione synthesis by TPMT in the presence of alkaline phosphatase. (B) Kinetics of the 6-methylmercaptopurine synthesis by TPMT. (C) Kinetic parameters of the kinetics shown in A and B.

FIG. 4 shows an HPLC chromatogram of the analysis of urothione after the incubation 500 μg liver extract (1.7 μg/μL) with 500 μM SAM, 250 μM DTT and 5 μM Moco in the presence and absence of 16 μM 3,4,5-triiodo benzoic acid (inhibitor).

FIG. 5 shows an HPLC chromatogram of the analysis of jukathione after the incubation of purified 3 ng/μL TPMT with 500 μM SAM, 250 μM DTT and 10 μM Moco in the presence and absence of 2.5 μM 3,4,5-triiodo benzoic acid (inhibitor).

FIG. 6 shows the in vitro synthesis of urothione in human liver protein extracts from individuals with a normal TPMT activity and persons with heterozygous mutations in the TPMT gene locus.

FIG. 7 shows the in vitro synthesis of urothione in protein extracts from RBCs from individuals with a normal TPMT activity (wild type) and persons with heterozygous mutations in the TPMT gene locus (heterozygous), and five persons with a homozygous mutation (homozygous) in the TPMT gene locus.

FIG. 8 illustrates the determination of the urothione content in the urine of individuals with a normal TPMT activity (wild type) and persons with heterozygous mutations in the TPMT gene locus (heterozygous), and two persons with a homozygous mutation (homozygous) in the TPMT gene locus.

### DETAILED DESCRIPTION OF THE INVENTION

### Structure of the Molybdenum Cofactor (Moco)

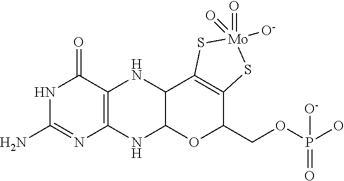

The catabolic final product and excretion product of Moco is urothione of the following structure (Goto and Sakurai, 1969):

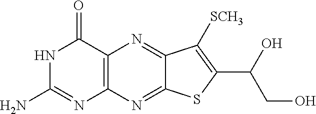 [SMILES: CSc1c(C(O)CO)sc2nc3nc(N)[nH]c(=O)c3nc12]

Urothione is also known under the IUPAC name 2-amino-7-(1,2-dihydroxyethyl)-6-methylsulfanyl-1H-thieno[3,2-g]pteridin-4-one or the term 2-amino-7-(1,2-dihydroxyethyl)-6-methylthio-1H-thieno[3,2-g]pteridin-4-one. It is a sulfur-containing pteridine derivative isolated from human urine.

It is still unknown today which enzymes take part in the transformation of Moco into urothione. The inventors have demonstrated for the first time that TPMT catalyzes the methylation of Moco. The present invention is based on this finding.

TPMT is capable of transforming Moco into urothione in an S-adenosylmethionine-dependent reaction in the presence of a phosphatase. The direct reaction product of TPMT is the phosphorylated form of urothione (jukathione), which is then transformed into urothione by a phosphatase not yet known, as shown in the following diagram. The steps that result in the formation of the substrate of 2-thiopurine methyltransferase (TPMT) are unknown. In an S-adenosylmethionine-dependent reaction TPMT forms jukathione.

The content of urothione in body fluids, e.g. urine, correlates with the activity of TPMT and can therefore be used as a reliable biomarker for determining the TPMT activity. Therefore, the determination of the urothione content can replace other less accurate methods, such as the determination of the activity by other substrates or genetic tests.

The use of urothione in the manner described makes possible:
the avoidance of thiopurine-associated side effects of any kind (e.g. myelosuppression, hepatotoxicity, pancreatitis, flu-like syndrome)
the prediction of the therapy response during thiopurine therapy for all indications of thiopurines, e.g. leukemia, chronic inflammatory bowel diseases (e.g. colitis ulcerosa, Crohn's disease), organ transplantations, autoimmune diseases, pulmonary fibrosis and others.
the avoidance of cisplatin-associated TPMT-mediated ototoxicity.

Thus, urothione and jukathione are very reliable biomarkers for the therapeutic monitoring under thiopurine therapy as an alternative for the determination of thiopurine metabolites in blood.

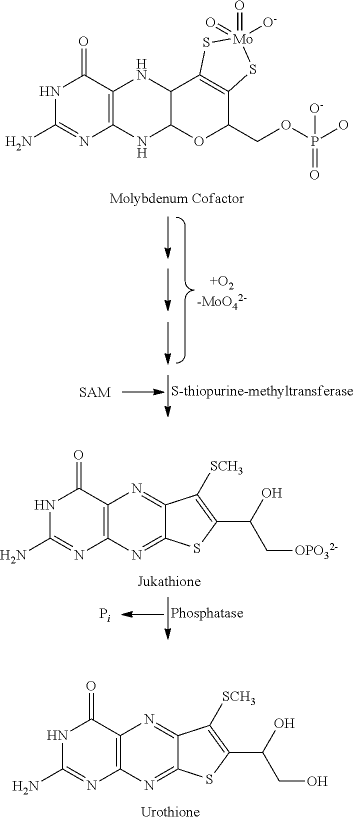

The following biochemical analyses prove that TPMT participates in the catabolism of urothione:

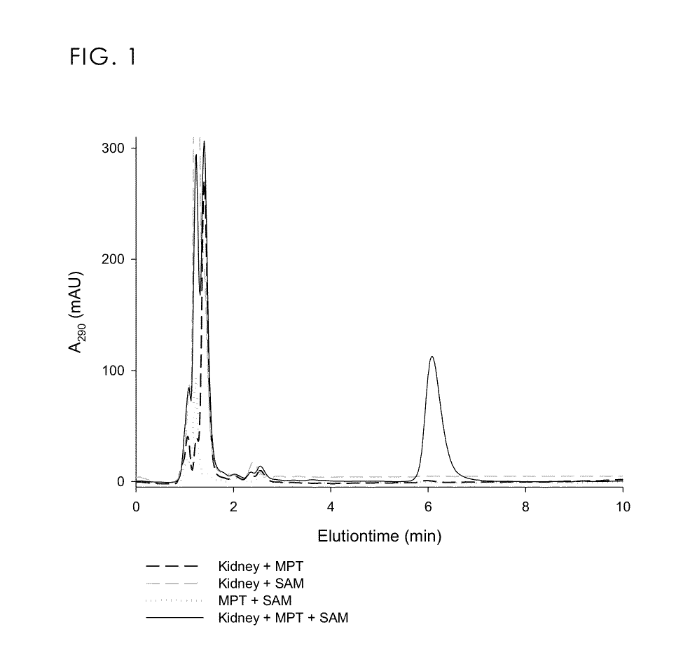

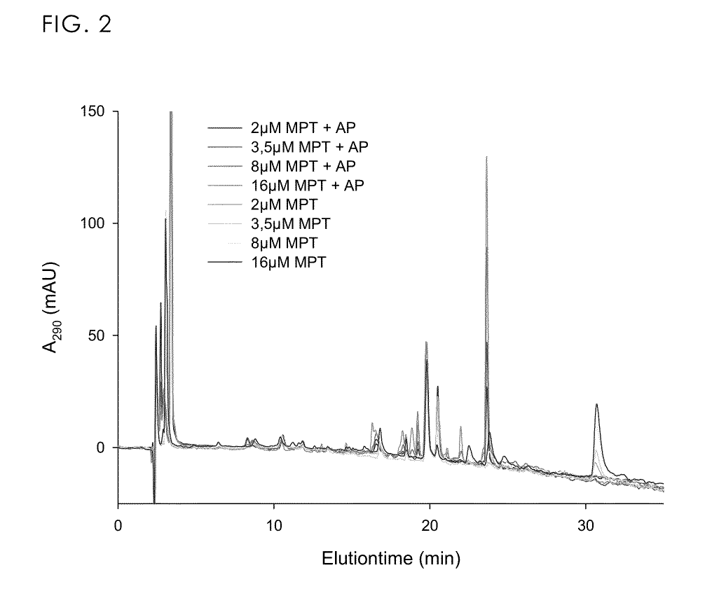

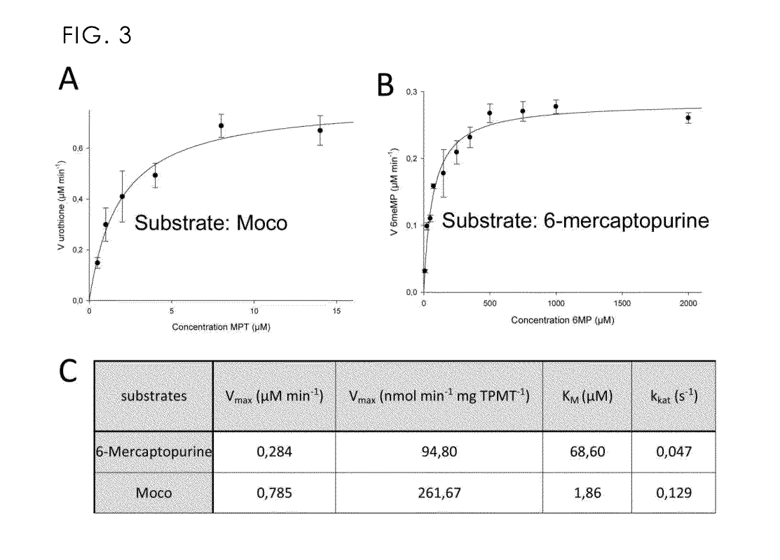

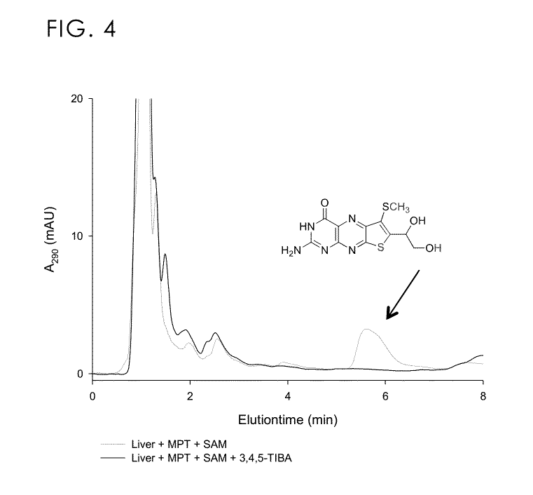

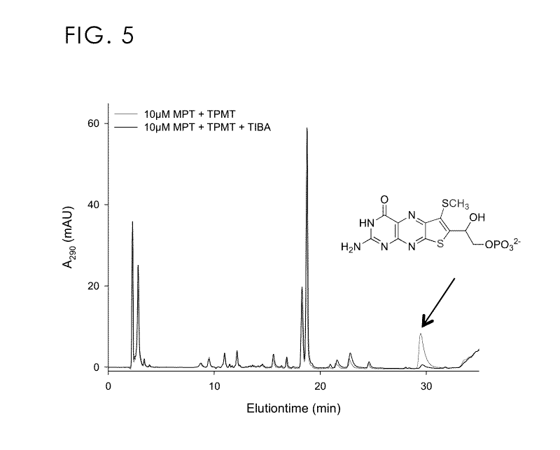

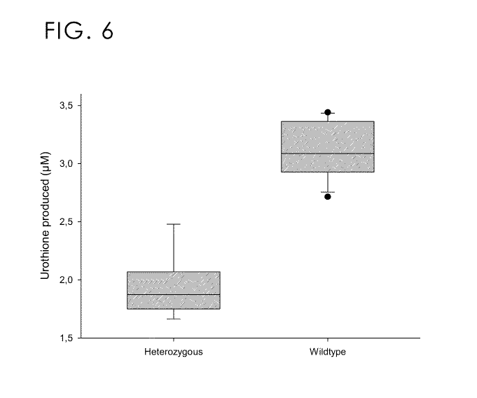

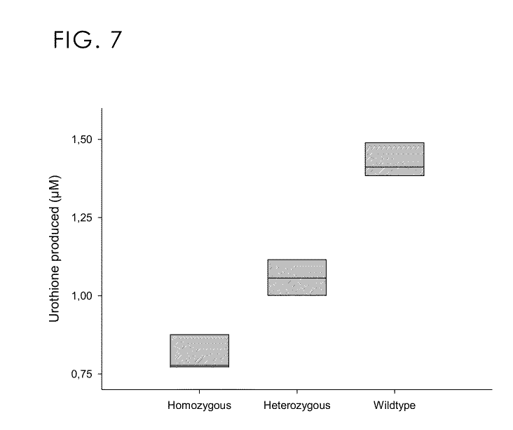

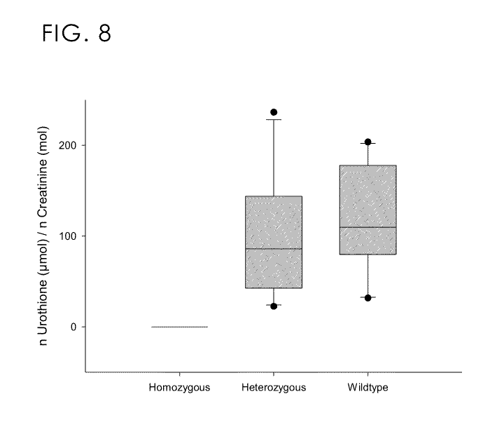

1. The in vitro synthesis of urothione in kidney protein extracts is dependent on S-adenosylmethionine and Moco. This is shown in FIG. 1. FIG. 1 shows an HPLC chromatogram of the analysis of urothione after the incubation of kidney extract (Kidney) with Moco (MPT), kidney extract (Kidney) with S-adenosylmethionine (SAM), Moco (MPT) with S-adenosylmethionine (SAM), and kidney extract (Kidney) with Moco (MPT) and S-adenosylmethionine (SAM). Only if all three components are incubated together can the formation of urothione be observed. Quantities used: 1.7 μg/μl kidney extract, 5 μM Moco, 500 μM SAM, 250 μM DTT in 0.1 M Tris/HCl pH7.5. The reaction was incubated for 4 h at 37° C. and then heat-denatured for 1 h at 60° for stopping and purified by means of QAE-Sepharose. The eluate was dried up and taken up in the HPLC solvent. HPLC analysis took place on a C4 Reprosil® 100 HPLC column, 150×2 mm, 5 μm particle size (Dr. Maisch GmbH) with 20 mM acetic acid and 15% methanol by isocratic elution.
2. The S-adenosylmethionine-dependent synthesis of urothione is dependent on the quantity of Moco and the presence of a phosphatase. In vitro, alkaline phosphatase is able to catalyze this reaction, as becomes clear from FIG. 2, which shows an HPLC chromatogram of the analysis of urothione after the incubation of purified TPMT with different quantities of Moco (MPT) in the absence and presence of alkaline phosphatase (+AP). The increase of the peak at 24 min elution time indicates the formation of urothione. The increase of the peak at 31 min elution time indicates the formation of jukathione. Quantities used: 20 ng/μl TPMT, 2-16 μM Moco, 500 μM SAM, 250 μM DTT in 0.1 M Tris/HCl pH7.5. The reaction was incubated for 1 h at 37° C. and then heat-denatured for 1 h at 60° for stopping. The eluate was dried up and taken up in the HPLC solvent. HPLC analysis took place on a YMC C18 Hydrosphere@ column, 250×4 mm, 5 μm particle size with 20 mM formic acid by elution in a methanol gradient (0-25% 20 min, 1 mL/min).
3. In vitro transformation of Moco by means of purified TPMT in urothione: The comparative analysis with recombinantly expressed and purified TPMT shows that Moco from a denatured Moco enzyme (human sulfite oxidase) as a substrate can be transformed into urothione in the presence of a phosphatase, as is shown in FIG. 3. The kinetic parameters of this reaction are: K_{M}=2 μM and k_{cat}=0.129 s⁻¹. Compared to 6-mercaptopurine as the substrate (K_{M}=69 μM and k_{cat}=0.047 s⁻¹), they exhibit a significantly more efficient substrate binding and catalytic reaction, as is to be expected from a physiological substrate. FIG. 3 illustrates steady state kinetics with 3 ng/μL purified TPMT and Moco (A) or 6-mercaptopurine (B) as a substrate. (C) Kinetic parameters of the kinetics shown in A and B.
4. The in vitro transformation of Moco by means of liver extracts into urothione in the presence and absence of the TPMT inhibitor 3,4,5-triiodo benzoic acid is shown in FIG. 4. The Figure shows an HPLC chromatogram of the analysis of urothione after the incubation 500 μg liver extract (1.7 μg/μl) with 500 μM SAM, 250 μM DTT and 5 μM Moco in the presence and absence of 16 μM 3,4,5-triiodo benzoic acid (inhibitor).
5. The in vitro transformation of Moco by purified TPMT into jukathione in the presence and absence of the TPMT inhibitor 3,4,5-triiodo benzoic acid is shown in FIG. 5. An HPLC chromatogram is shown of the analysis of jukathione after the incubation of purified 1 ng/μL TPMT with 500 μM SAM, 250 μM DTT and 10 μM Moco in the presence and absence of 2.5 μM 3,4,5-triiodo benzoic acid (inhibitor).
6. FIG. 6 shows the in vitro synthesis of Moco in human liver protein extracts from individuals with a normal TPMT activity. Compared to persons with heterozygous mutations in the TPMT gene locus, an activity in the urothione synthesis that is reduced to about 60% is observed.
7. The in vitro transformation of Moco by protein extracts from RBCs of individuals with a normal TPMT activity, compared with persons with heterozygous mutations in the TPMT gene locus, shows an activity in the urothione synthesis that is reduced to about 70%. As is apparent from FIG. 7, a urothione synthesis reduced by 50% can be detected in five persons with homozygous mutations in the TPMT gene locus. What is depicted is the in vitro synthesis of urothione in protein extracts from RBCs from individuals with a normal TPMT activity (wild type) and persons with heterozygous mutations in the TPMT gene locus (heterozygous), and one person with a homozygous mutation (homozygous) in the TPMT gene locus.
8. The determination of the urothione content in the urine of individuals with a normal TPMT activity, in comparison with persons with heterozygous mutations in the TPMT gene locus, shows no significant differences in the urothione content, while no urothione could be detected in two persons with homozygous mutations in the TPMT gene locus (FIG. 8). What is shown is the determination of the urothione content in individuals with a normal TPMT activity (wild type) and persons with heterozygous mutations in the TPMT gene locus (heterozygous), and two persons with homozygous mutations (homozygous) in the TPMT gene locus.

### Examples

### Material and Method

Urothione detection: 10 μM Moco (isolated from heat-denaturated human sulfite oxidase) are incubated in 50 mM Tris/HCl, pH 7.2, 1 mM SAM, 250 μM dithiothreitol and 1.7 μg/μL liver protein extract or 3.4 μg/μL RBCs for 4 h at 37° C. After adding 33 units of alkaline phosphatase, 20 mM MgCl₂ and 0.1 M Tris/HCl, pH 8.3, incubation is carried out for at least 4 h (37° C.) and the reaction is stopped by 15 minutes of incubation at 80° C. The HPLC analysis of urothione took place on a YMC C18 Hydrosphere®, 250×4 mm column with a 5 μm particle size in 20 mM formic acid and elution in a methanol gradient (0-25% 20 min, 1 mL/min).

A final concentration of 3 ng/μL enzyme was used for the reaction kinetics with purified TPMT. The reaction was stopped with 3,4,5-triiodo benzoic acid. The analysis took place as described above.

In order to detect urothione in urine, a two-stage solid phase extraction with Florisil® (500 mg matrix/mL urine, elution with 50% acetone) and an aminopropyl matrix was carried out (500 mg matrix/mL urine, elution with 20 mM acetic acid). A C18 Reprosil® 100, 250×3 mm column with 5 μm particles (Dr. Maisch GmbH) was used for the subsequent HPLC analysis. 20 mM formic acid served as eluent, and elution took place in a methanol gradient (0-25% 20 min, 1 mL/min). Creatinine was determined according to Vasiliades.

### INDUSTRIAL APPLICABILITY

According to the invention, the urothione level is determined in vitro in body fluids or cellular extracts and correlated with the TPMT activity. Using the data obtained in this manner, cut-off values for urothione levels can be determined for the stratification of TMPT-deficient individuals, individuals with a reduced TPMT activity, individuals with a normal TPMT activity and individuals with an extremely high level of TPMT activity. Then, these cut-off values serve for the dose-adjusted therapy of patients with thiopurines, in analogy to the current procedure using the measurement of the TPMT activity in RBCs or the genetic diagnostics for the TPMT variants.

What is claimed is:

1. A diagnostic in vitro method for determining the activity of thiopurine S-methyltransferase (TPMT) in individuals to be examined, characterized in that the content of urothione and/or jukathione in body fluids is determined.

2. The method according to claim 1, characterized in that the content of urothione and/or jukathione is determined in urine, serum and/or plasma.

3. A diagnostic in vitro method for determining the TPMT activity in cellular extracts of test subjects of any origin, characterized in that molybdenum cofactor (Moco) and/or decomposition stages of Moco are used as substrates for the reaction and the formed quantity of jukathione and/or urothione is determined.

4. The method according to claim 3, characterized in that the cellular extracts originate from fibroblasts, erythrocytes and/or biopsy material.

5. The method according to claim 1, wherein the content of urothione and/or jukathione in the body fluid serves for determining the optimum dose of thiopurines in patients.

6. A diagnostic in vitro method, wherein jukathione is determined in body fluids and/or cellular extracts and serves as a biomarker for an increased susceptibility with regard to one or more particular diseases that are characterized by a change in the activity of molybdenum-containing enzymes.

7. The diagnostic in vitro method according to claim 6, wherein the molybdenum-containing enzyme is sulfite oxidase.
